(19) United States
(12) Patent Application Publication
(10) Pub. No.: US 2026/0110609 A1
(43) Pub. Date: Apr. 23, 2026

(54) STRESS-STRAIN PREDICTION METHOD FOR GAP-GRADED SOILS BASED ON COUPLING OF DISCRETE ELEMENT METHOD AND MACHINE LEARNING

(71) Applicant: Zhejiang University of Technology, Hangzhou (CN)

(72) Inventors: Shanlin XU, Hangzhou (CN); Lingkai HU, Hangzhou (CN); Honglei SUN, Hangzhou (CN); Yanzhen ZHU, Hangzhou (CN); Chenhao YE, Hangzhou (CN)

(73) Assignee: Zhejiang University of Technology, Hangzhou (CN)

(21) Appl. No.: 19/357,071

(22) Filed: Oct. 13, 2025

(30) Foreign Application Priority Data
Aug. 13, 2024 (CN) 202411106895.7

(51) Int. Cl. G01N 3/02; G01N 3/08; G01N 33/24; G06N 5/01

(52) U.S. Cl. CPC G01N 3/02; G01N 3/08; G01N 33/24; G06N 5/01

(57) ABSTRACT

The present disclosure relates to stress-strain prediction method for gap-graded soils based on coupling of the discrete element method and machine learning. The method includes the following steps: S1. obtaining baseline stress-strain data; S2. establishing and verifying a discrete element model; S3. establishing discrete element specimens of gap-graded soils with different gradations; S4. obtaining stress-strain data of the different gap-graded soils; S5. establishing a raw database; S6. partition the raw database; S7. establishing a random forest model; S8. training the random forest model to obtain a stress prediction model; S9. evaluating the trained random forest model; S10. predicting stress data of the gap-graded soils. This method can quickly predict the stress-strain curve of a gap-graded soil directly with its particle size ratio and fines content, improving efficiency.

### CROSS-REFERENCE TO RELATED APPLICATIONS

The application claims priority to Chinese patent application No. 202411106895.7, filed on Aug. 13, 2024, the entire contents of which are incorporated herein by reference.

### TECHNICAL FIELD

The present disclosure relates to the technical field of stress-strain prediction, and in particular to a stress-strain prediction method for gap-graded soils based on coupling of the discrete element method and machine learning.

### BACKGROUND

Under actions of landslides and debris flows, coarse particles (sand and gravel) and fine particles (silt) intermix, forming gap-graded soil particle mixtures. Gap-graded soils exhibit favorable drainage, internal stability and compactability, and are widely used in hydraulic structures such as embankments and dams. Research on the physical and mechanical properties of gap-graded soils has become a critical issue in engineering practice. Currently, numerical values for the mechanical properties and design parameters of soil masses rely almost entirely on in-situ geotechnical tests and laboratory geotechnical tests, which incur high economic and time costs. Consequently, it is of significant importance to establish efficient and reliable constitutive prediction models for gap-graded soils.

### SUMMARY

In view of the shortcomings in the prior art, the present disclosure provides a stress-strain prediction method for gap-graded soils based on coupling of the discrete element method and machine learning to solve the technical problems. This method can quickly predict the stress-strain curve of a gap-graded soil directly with its particle size ratio and fines content.

The object of the present disclosure is accomplished by the following technical solutions. A stress-strain prediction method for gap-graded soils based on coupling of the discrete element method and machine learning includes the following steps:
S1. obtaining baseline stress-strain data,
wherein a reference specimen is consolidated and saturated, and sheared using a triaxial testing apparatus, then the axial force is measured by a proving ring, and the deformation on the specimen surface is recorded by a digital image measurement system, such that the stress-strain curve of the specimen is obtained, yielding the baseline stress-strain data;
S2. establishing and verifying a discrete element model,
wherein using a specimen with the same gradation as in S1, a conventional triaxial compression test model is established by using the discrete element method to perform compression simulation, the results are compared with the baseline stress-strain data obtained in S1 to verify the accuracy of the conventional triaxial compression model established using the discrete element method and to determine the particle parameters and loading conditions in the discrete element simulation model;
S3. establishing discrete element models of gap-graded soils with different gradations,
wherein the particle gradation in the simulation specimens is adjusted to establish discrete element specimens with different fines contents and particle size ratios;
S4. obtaining stress-strain data of the specimens with different gradations,
wherein isotropic consolidation and in turn conventional triaxial compression simulation tests are performed on the discrete element models with different gradations from S3 to obtain the stress-strain data for the different gradations;
S5. establishing a raw database,
wherein the stress-strain data obtained from the simulation in S4 is preprocessed and the corresponding characteristic values of fines content and particle size ratio are added, so as to establish the raw database;
S6. partitioning the raw database,
wherein the raw database is partitioned into a training set, a validation set and a test set with a data ratio of 8:1:1;
S7. establishing a random forest model,
wherein the inputs to the random forest model include strain, particle size ratio, and fines content, and the output is axial stress;

S8. training the random forest model to obtain a stress prediction model,
wherein hyperparameter optimization via the Bayesian method is performed using the validation set data to obtain and save the optimal-parameter stress prediction model;

S9. evaluating the trained random forest model,
wherein the trained stress prediction random forest model from S8 is verified using the test set data, the prediction data so obtained is compared with the raw data, and the model performance is computed to evaluate the accuracy of the obtained random forest model; and

S10. predicting stress data of gap-graded soils,
wherein different gradation parameters and axial strains of the gap-graded soils, for which stress-strain data needs to be predicted, are input into the trained stress prediction model from S8 to obtain the predicted stress data of the gap-graded soils.

The effects of the present disclosure are as follows.
(1) This method simulates conventional triaxial compression tests based on the discrete element method to obtain the stress-strain response of gap-graded soils with different gradations, and employs a machine learning and data-driven method to establish the constitutive relationship of gap-graded soils.
(2) As traditional constitutive models are complex and involve numerous parameters, limiting their application in describing the stress-strain relationship of soils, the machine learning method adopted in this approach, when applied to the constitutive modeling of gap-graded soils, requires no assumptions. Machine learning can directly learn the stress-strain relationship from the raw data. As the data set increases, the machine learning-based model shows significant improvement in both prediction accuracy and range of application.

### BRIEF DESCRIPTION OF DRAWINGS

FIG. 1 is a flowchart of the technical methodology in the present disclosure;

FIG. 2 is a schematic diagram of model verification in the present disclosure;

FIG. 3 is a schematic diagram of particle generation and isotropic consolidation in the present disclosure;

FIG. 4 is a schematic diagram of simulated conventional triaxial compression in the present disclosure;

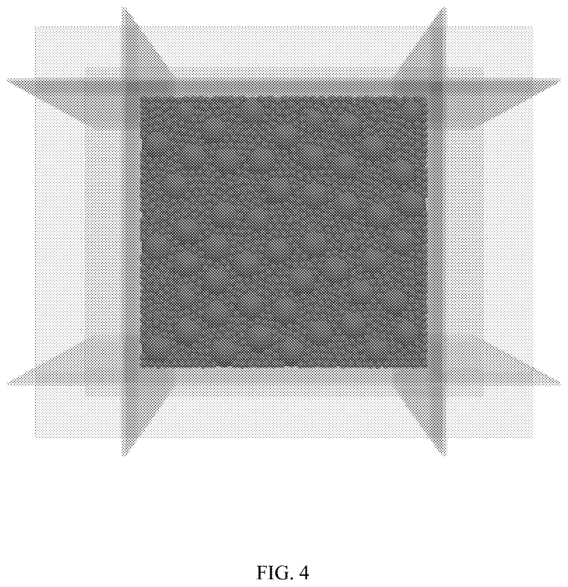

FIG. 5 is a schematic diagram of data preprocessing in the present disclosure;

FIG. 6 is a schematic diagram of hyperparameter optimization in the present disclosure;

FIG. 7 is a schematic diagram showing a comparison between predicted values and true values in the present disclosure; and

FIG. 8 is a schematic diagram showing the predictions for different particle size ratios and fines contents in the present disclosure.

### DETAILED DESCRIPTION OF THE EMBODIMENTS

To further explain the technical means adopted by the present disclosure to achieve its predetermined purpose, and to explain the effects of the technical means, specific implementations, structures, features and effects of the present disclosure will be described in detail below in conjunction with the accompanying drawings and preferable embodiments.

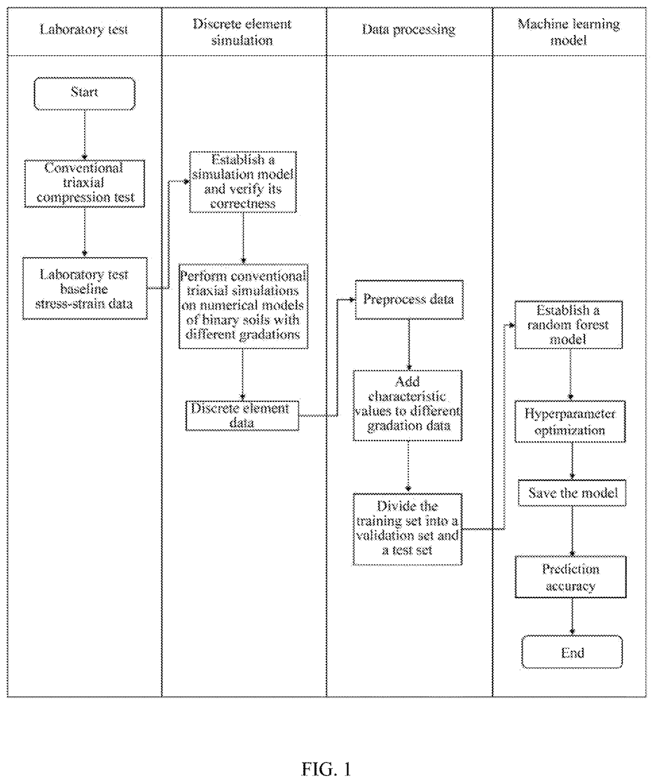

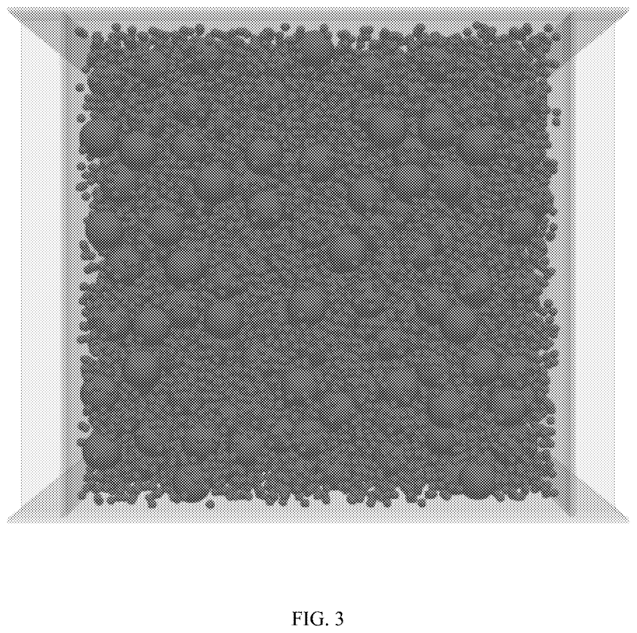

Referring to FIGS. 1 to 3, a stress-strain prediction method for gap-graded soils based on coupling of the discrete element method and machine learning is provided and has the following steps.

S1. Obtain baseline stress-strain data.

A reference specimen is consolidated and saturated, and then sheared using a triaxial testing apparatus. Then the axial force is measured by a proving ring and the deformation on the specimen surface is recorded by a digital image measurement system. The stress-strain curve of the specimen is obtained, yielding the baseline stress-strain data. Here, the above-mentioned specimen is preferably a quartz sand specimen with a relative density of 50% that is fully consolidated and saturated and then sheared in the triaxial testing apparatus at a constant strain rate of 0.2%/min.

S2. Establish and verify a discrete element simulation model.

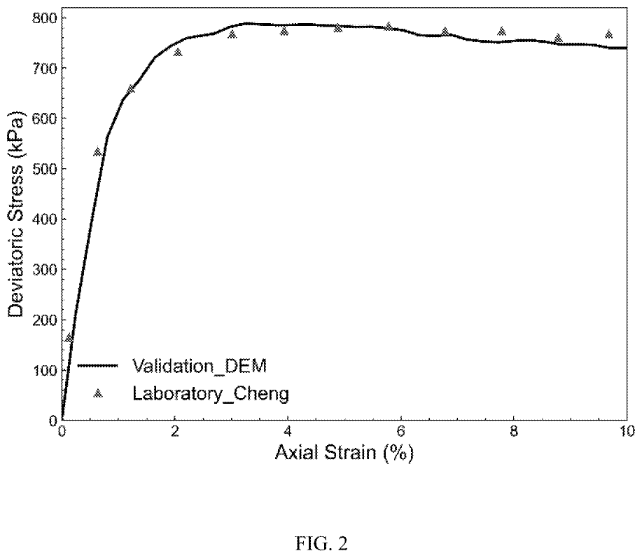

Using a specimen with the same gradation as in S1, a conventional triaxial compression model is established using the discrete element method. The output of this model is compared with the baseline stress-strain data obtained in S1 to verify the correctness of the discrete element simulation model and to determine the particle parameters and loading conditions in the discrete element simulation model. The comparison of the stress-strain curves between the numerical simulation and the laboratory test is shown in FIG. 2.

Specifically, in S2-1, when simulating the conventional triaxial test using the discrete element method, quasi-static conditions need to be satisfied, and therefore the moving velocity of the upper and lower walls should not be excessively high. However, if the loading velocity is excessively low, the computational cost would be greatly increased. Therefore, the loading velocity is preferably 0.0002 n's in the discrete element simulation.

In S2-2, to maintain computational stability, the actual time step used in the discrete element simulation must be less than the critical time step, and the time step is preferably 2e⁻⁷ s.

In S2-3, to avoid boundary effects, the ratio of the specimen length to the coarse particle diameter before conventional triaxial compression is 10.

In S2-4, the numerical parameters for the particles in the discrete element simulation include a Young's modulus of 70 GPa, a density of 2650 kg/m³, a Poisson's ratio of 0.3, a coefficient of restitution 0.2, a sliding friction coefficient 0.5, and a rolling friction coefficient 0.1 of the simulated particles.

S3. Establish discrete element models of gap-graded soils with different gradations. The particle gradation in the simulation model specimens is adjusted to establish discrete element specimens with different fines contents and particle size ratios. The specimen particles consist of coarse particles and fine particles.

Specifically, specimens with specified fines content and particle size ratio can be generated within a 24 mm×24 mm×24 mm simulation region, where the position of each particle is random. Here, the particle size ratio is determined by Formula (1), and the fines content is determined by Formula (2):

$$SR = \frac{{d}_{max}}{{d}_{min}} \qquad ( 1 )$$

$$FC = \frac{{m}_{fine}}{{m}_{all}} \qquad ( 2 )$$

In the formulas above, SR denotes the particle size ratio, dₘₐₓ and dₘᵢₙ denote the diameters of coarse particles and fine particles respectively, FC denotes the fines content, and m_{fine} and mₐₗₗ denote the mass of fine particles and the mass of all particles respectively.

S4. Obtain stress-strain data for different gradations. Isotropic consolidation and in turn conventional triaxial compression test simulations are performed on the discrete element models with different gradations from S3 to obtain the stress-strain data for the different gradations.

Specifically, the isotropic consolidation test simulation includes placing the specimen within six servo-controlled walls, setting the sliding friction coefficient to zero, and simultaneously moving the six servo-controlled walls slowly toward the center until the specified initial confining pressure of 100 kPa is reached and stabilized, thereby generating a dense and isotropic specimen.

The isotropic specimen is then subjected to a conventional triaxial compression simulation test. The inter-particle friction coefficient is adjusted to 0.5 and the rolling friction coefficient is set to 0.1, to approximately account for the influence of irregular particle shapes. During loading, σᵢ is achieved by moving the upper and lower walls at a constant velocity, while the confining pressures σ₂ and as are maintained at the specified value of 100 kPa by the servo-controlled wall units. The stress-strain data for the different gradations are thus obtained.

S5. Establish the raw database. The stress-strain data obtained from the simulation in S4 is preprocessed to obtain simplified stress-strain curves. The corresponding characteristic values of fines content and particle size ratio are added to establish the raw database.

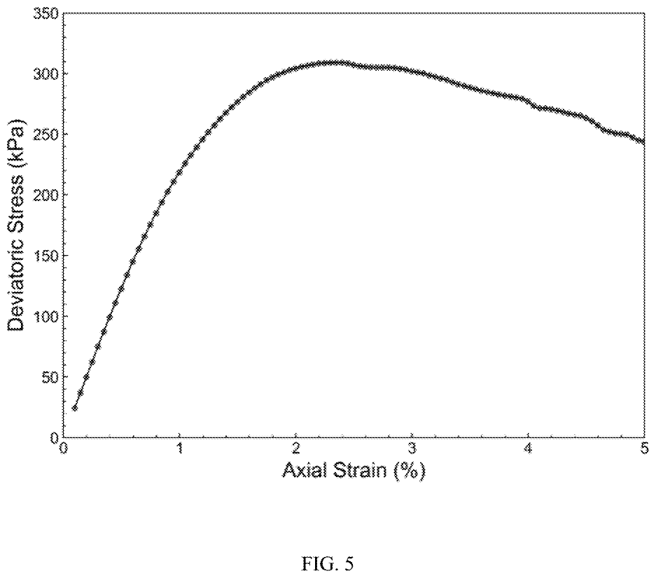

Specifically, in S5-1, if the untreated deviator stress-strain curves are used as input and output data for the neural network, the burden caused by model evaluation would be significantly increased, consuming substantial computational resources. In view of this, the present disclosure preprocesses and thus simplifies the stress-strain curves. Different stress-strain curves are represented using the same abscissa values and different ordinate values, resulting in a stress-strain curve defined by two-dimensional coordinates. The abscissa of the curve is divided equally into 100 segments, and 100 points corresponding to the fixed, equally spaced abscissa values are taken to characterize a set of stress-strain curves with different particle size ratios and fines contents (as shown in FIG. 5, points are taken at an interval of 0.05).

In S5-2, a stress-strain curve represented by 100 two-dimensional coordinates is obtained from S5-1, and then the characteristic parameters of fines content and particle size ratio are added as prefixes to each coordinate, resulting in 100 four-dimensional coordinates and thus establishing the raw database.

S6. Partition the raw database. The raw database is partitioned into a training set, a validation set, and a test set with a data ratio of 8:1:1.

Specifically, the raw dataset is partitioned using a Python script that imports the “train_test_split” function from the “sklearn.model_selection” module, with “random_state=42” set to ensure reproducible products.

S7. Establish a random forest model. Here, the inputs to the random forest model include strain, particle size ratio and fines content, and the output is strain.

Specifically, the random forest is an ensemble technique that includes multiple decision trees. Its output is the average of all trees. This method can effectively reduce overfitting and improve the generalization ability of the model. Establishing the random forest model involves the following steps.

In S7-1, samples are randomly selected from the training set.

In S7-2, n decision trees are generated. Using a Python script, the “RandomForestRegressor” class is imported from the “sklearn.ensemble” module, and “RandomForestRegressor” is invoked to create a random forest regressor.

In S7-3, the data subset for each decision tree is generated by sampling based on the bagging concept.

In S7-4, finally, the prediction results of all decision trees are averaged to obtain the final prediction.

The convergence of the random forest is expressed by the following Formula (3):

$$mg ( x , y ) = {av}_{n} I ( {f}_{n} ( x ) = y ) - {max}_{j = y} {av}_{n} I ( {f}_{n} ( x ) = j ) \qquad ( 3 )$$

where fₙ(x) represents a decision tree, n represents the number of trees in the random forest, I(.) is the indicator function, and avₙ is the average value of the function. A larger mg(x, y) value indicates more accurate predictions from the random forest.

S8. Train the random forest model to obtain a stress prediction model. Specifically, Bayesian hyperparameter optimization is performed using the validation set to obtain and save the optimal-parameter stress prediction model.

Bayesian hyperparameter optimization is implemented by using the “skopt” module of the “Scikit-Optimize” library to call the “BayesSearchCV” function for Bayesian optimization. Compared to traditional grid search and random search, Bayesian optimization considers previous evaluation results, can explore the parameter space more efficiently, and typically requires fewer iterations to find better results. Here, the Bayesian theorem is expressed as follows:

$$P ( H \vert E ) = \frac{P ( E \vert H ) P ( H )}{P ( E )} \qquad ( 4 )$$

where P(H|E) is the likelihood of the target H given E; P(E|H) is the likelihood of observing E given hypothesis H; P(H) is the prior probability of hypothesis H without observing evidence E; and P(E) is the marginal likelihood of evidence E.

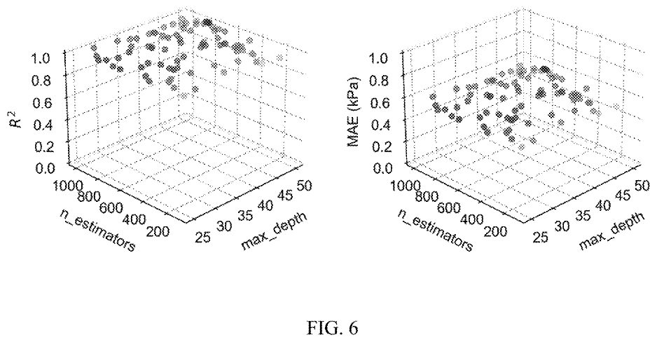

Hyperparameter tuning is performed for “n estimators” and “max depth”. The results of the Bayesian optimization are shown in FIG. 6 and Table 1.

TABLE 1

| Parameter name | Search range | Result |
|---|---|---|
| n_estimators | [100, 1000] | 810 |
| max_depth | [1, 60] | 50 |

S9. Evaluate the trained random forest model. Based on the hyperparameters determined by the Bayesian method in S8, the test set data is input into the trained stress prediction model from S8 to obtain prediction data, and the model performance is calculated. The random forest model is evaluated using the test set. Here, the calculation formulas for the goodness-of-fit R² and mean absolute error MAE as model performance metrics are as follows:

$${R}^{2} = \frac{SSR}{SST} = \frac{{\sum }_{i = 1}^{n} {( {\hat{y}}_{i} - \overline{y} )}^{2}}{{\sum }_{i = 1}^{n} {( {y}_{i} - \overline{y} )}^{2}} \qquad ( 5 )$$

$$MAE = \frac{1}{n} {\sum }_{i = 1}^{n} \vert {y}_{i} - {\hat{y}}_{i} \vert \qquad ( 6 )$$

Where ŷ is the predicted value; y is the experimental value; n is the number of samples.

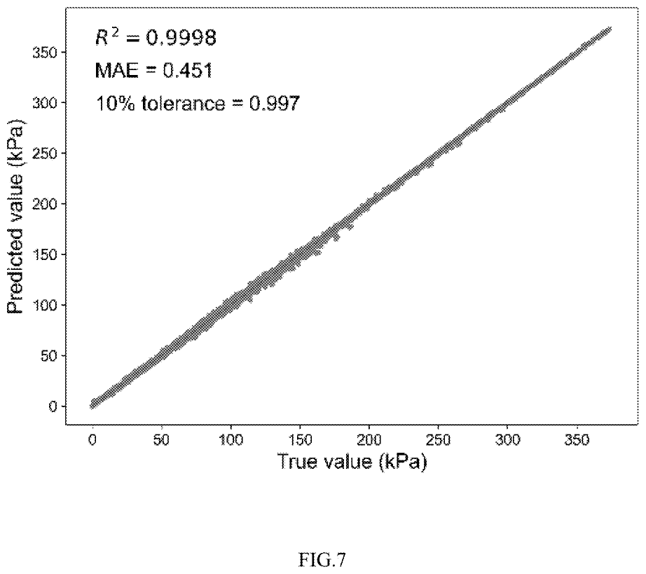

As shown in FIG. 7, the predicted values based on the prediction parameters of the random forest model are compared against the true values. In FIG. 7, R², MAE and 10% tolerance represent the coefficient of determination, mean absolute error and prediction accuracy within a 10% tolerance error, respectively. The abscissa and ordinate represent the true value and predicted value of the deviator stress parameter, respectively. When a scatter point in FIG. 7 is closer to the diagonal line, it indicates that the predicted value and true value for the data corresponding to that point are closer to each other. The scatter points of the random forest model are all relatively close to the diagonal line.

S10. Predict stress data of gap-graded soils. The different gradation parameters and axial strains of the gap-graded soils, for which stress-strain data needs to be predicted, are input into the trained stress prediction model from S8 to obtain the predicted stress data for the gap-graded soils.

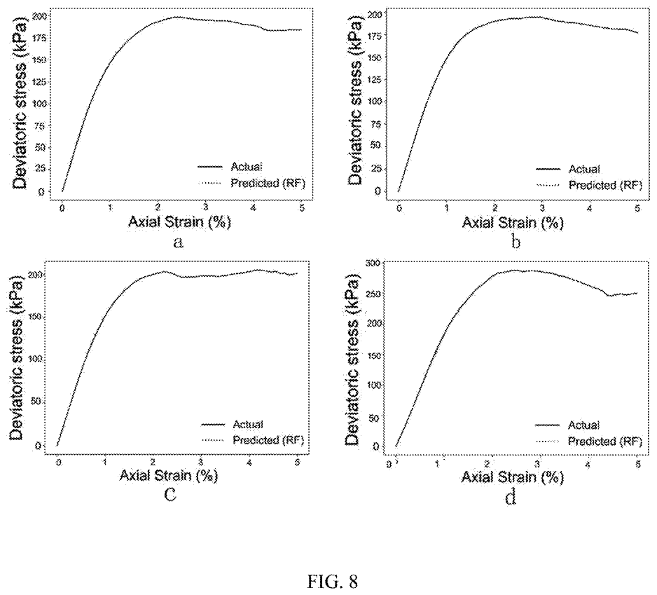

As shown in FIG. 8, (a) shows the result for particle size ratio 2 and fines content 22.5%; (b) shows the result for particle-size ratio 3 and fines content 12.5%; (c) shows the result for particle size ratio 4 and fines content 10%; (d) shows the result for particle size ratio 6 and fines content 22.5%. It can be seen that the random forest model demonstrates good performance in predicting the stress-strain curves of gap-graded soils with different gradations, and the predicted values are largely consistent with the experimental values.

The above description only illustrates preferable embodiments of the present disclosure and is not intended to limit the present disclosure in any form. Although the present disclosure has been disclosed above in the form of preferable embodiments, it is not intended to defme the present disclosure. Using the technical contents disclosed above, those of ordinary skill in the art can make some changes or modifications as equivalent embodiments, without departing from the scope of the technical solutions of the present disclosure. However, any simple modifications, equivalent changes and modifications made to the above embodiments based on the technical spirit of the present disclosure without departing from the content of the technical solutions of the present disclosure still fall within the scope of the technical solutions of the present disclosure.

What is claimed is:

1. A stress-strain prediction method for gap-graded soils based on coupling of the discrete element method and machine learning, the prediction method comprising the following steps:
S1. obtaining baseline stress-strain data,
wherein a reference specimen is consolidated and saturated, and sheared using a triaxial testing apparatus, then the axial force is measured by a proving ring, and the deformation on the specimen surface is recorded by a digital image measurement system, such that the stress-strain curve of the specimen is obtained, yielding the baseline stress-strain data;
S2. establishing and verifying a discrete element model,
wherein using a specimen with the same gradation as in S1, a conventional triaxial compression model is established by using the discrete element method to perform compression simulation, the results are compared with the baseline stress-strain data obtained in S1 to verify the accuracy of the conventional triaxial compression model established using the discrete element method and to determine the particle parameters and loading conditions in the discrete element simulation model;
S3. establishing discrete element models of gap-graded soils with different gradations,
wherein the particle gradation in the simulation specimens is adjusted to establish discrete element specimens with different fines contents and particle size ratios;
S4. obtaining stress-strain data of the specimens with different gradations,
wherein isotropic consolidation and in turn conventional triaxial compression simulation tests are performed on the discrete element models with different gradations from S3 to obtain the stress-strain data for the different gradations;
S5. establishing a raw database,
wherein the stress-strain data obtained from the simulation in S4 is preprocessed and the corresponding characteristic values of fines content and particle size ratio are added, so as to establish the raw database;
S6. partitioning the raw database,
wherein the raw database is partitioned into a training set, a validation set and a test set with a data ratio of 8:1:1;
S7. establishing a random forest model,
wherein the inputs to the random forest model include strain, particle size ratio, and fines content, and the output is strain;
S8. training the random forest model to obtain a stress prediction model,
wherein hyperparameter optimization via the Bayesian method is performed using the validation set data to obtain and save the optimal-parameter stress prediction model;
S9. evaluating the trained random forest model,
wherein the trained stress prediction random forest model from S8 is verified using the test set data, the prediction data so obtained is compared with the raw data, and the model performance is computed to evaluate the accuracy of the obtained random forest model; and
S10. predicting stress data of the gap-graded soils,
wherein different gradation parameters and axial strains of the gap-graded soils, for which stress-strain data needs to be predicted, are input into the trained stress prediction model from S8 to obtain the predicted stress data of the gap-graded soils.

2. The stress-strain prediction method for gap-graded soils based on coupling of the discrete element method and machine learning of claim 1, wherein particle gradations are configured in S3 to prepare particle specimens with different fines contents and particle size ratios, the specimen particles comprise coarse particles and fine particles, the particle size ratio is determined by Formula (1), and the fines content is determined by Formula (2):
$$SR = \frac{{d}_{max}}{{d}_{min}} \qquad ( 1 )$$
$$FC = \frac{{m}_{fine}}{{m}_{all}} \qquad ( 2 )$$
wherein SR denotes the particle size ratio, dₘₐₓ and dₘᵢₙ, denote the diameters of coarse particles and fine particles respectively, FC denotes the fines content, and m_{fine} and mₐₗₗ denote the mass of fine particles and the mass of all particles respectively.

3. The stress-strain prediction method for gap-graded soils based on coupling of the discrete element method and machine learning of claim 1, wherein in the conventional triaxial compression test simulation using the discrete element method in S2, the numerical parameters for the sand simulated by the discrete element method comprise a Young's modulus of 70 GPa, a density of 2650 kg/m³, a Poisson's ratio of 0.3, a coefficient of restitution 0.2, a sliding friction coefficient 0.5, a rolling friction coefficient 0.1, a loading velocity of 0.0002 m/s in the discrete element simulation and a time step of 1e⁻⁷ s of the simulated particles.

4. The stress-strain prediction method for gap-graded soils based on coupling of the discrete element method and machine learning of claim 1, wherein in the isotropic consolidation test in S4, a cubic specimen is placed within six servo-controlled walls, the inter-particle sliding friction coefficient is set to zero, and the six servo-controlled walls are moved simultaneously toward the center slowly at a velocity of 0.025 m/s until the specified initial confining pressure of 100 kPa is reached and stabilized, thereby generating a dense and isotropic specimen.

5. The stress-strain prediction method for gap-graded soils based on coupling of the discrete element method and machine learning of claim 4, wherein the isotropic specimens in S4 undergo the conventional triaxial compression simulation tests with the inter-particle friction coefficient adjusted to 0.5 and the rolling friction coefficient set to 0.1, during loading, by moving the upper and lower walls at a constant velocity, the confining pressure is maintained at the specified value of 100 kPa via the servo-controlled walls in the horizontal direction to obtain the stress-strain data of the specimens with different gradations.

6. The stress-strain prediction method for gap-graded soils based on coupling of the discrete element method and machine learning of claim 1, wherein the stress-strain data is preprocessed for simplification in S5, such that different stress-strain curves are represented using the same abscissa values and different ordinate values to obtain a stress-strain curve defined by two-dimensional coordinates and the characteristic parameters of fines content and particle size ratio are then added as prefixes to each coordinate, resulting in four-dimensional coordinates.

7. The stress-strain prediction method for gap-graded soils based on coupling of the discrete element method and machine learning of claim 1, wherein the step of establishing the random forest model in S7 comprises the following steps: 1) selecting samples from the training set randomly; 2) generating n decision trees, with the data subset in each decision tree generated by sampling based on the bagging concept; 3) obtaining the final prediction by averaging the prediction results of all decision trees.

8. The stress-strain prediction method for gap-graded soils based on coupling of the discrete element method and machine learning of claim 7, wherein the convergence of the random forest is expressed by the following Formula (3):
$$mg ( x , y ) = {av}_{n} I ( {f}_{n} ( x ) = y ) - {max}_{j = y} {av}_{n} I ( {f}_{n} ( x ) = j ) \qquad ( 3 )$$
wherein fₙ(x) represents a decision tree, n represents the number of trees in the random forest, I(.) is the indicator function, and avₙ is the average value of the function, a larger mg (x, y) value indicates more accurate predictions from the random forest.

9. The stress-strain prediction method for gap-graded soils based on coupling of the discrete element method and machine learning of claim 1, wherein for the hyperparameter optimization using the Bayesian method in S8, the Bayesian theorem is expressed as follows:
$$P ( H \vert E ) = \frac{P ( E \vert H ) P ( H )}{P ( E )} \qquad ( 4 )$$
wherein P(H|E) is the likelihood of the target H given E; P(E|H) is the likelihood of observing E given the hypothesis H; P(H) is the prior probability of hypothesis H without observing evidence E; and P(E) is the marginal likelihood of evidence E.

10. The stress-strain prediction method for gap-graded soils based on coupling of the discrete element method and machine learning of claim 1, wherein the specimen in S1 is a quartz sand specimen with a relative density of 50% that is fully consolidated and saturated and then sheared in the triaxial testing apparatus at a constant strain rate of 0.2%/min.
